(19) United States
(12) Patent Application Publication
(10) Pub. No.: US 2006/0287384 A1
(43) Pub. Date: Dec. 21, 2006

(54) Lipoic acid concentrate

(76) Inventor: Dariush Behnam, Rossdorf (DE)

Correspondence Address: JACOBSON HOLMAN PLLC, 400 SEVENTH STREET N.W., SUITE 600, WASHINGTON, DC, 20004, US

(21) Appl. No.: 10/572,918

(22) PCT Filed: Aug. 18, 2005
(86) PCT No.: PCT/EP05/08940

§ 371 Date: Mar. 21, 2006

(30) Foreign Application Priority Data
Aug. 18, 2004 (DE) 1020040401780

(51) Int. Cl. A61K 31/385; A61K 31/22; A61K 31/122

(52) U.S. Cl. 514/440; 514/547; 514/690

(57) ABSTRACT

A water-free concentrate, consisting of ubichinon Q₁₀, a medium-chained triglyceride or triglyceride mixture, α-lipoic acid and/or its derivative as well as one or more emulgators with HLB value between 9 and 19 permitted according to the foodstuff or drug laws has been described.

The invention relates to an α-lipoic acid concentrate according to DE 101 08 614 A1.

Since the study by K. Reff et al. (Diabetes und Stoffwechsel [metabolism], 1996, 5/3, Suppl. (59-63)), it is known that in overweight patients, administration of α-lipoic acid alleviates their problems. Further, a study conducted on rats by Min-Seon Kim et al. (NATURE MEDICINE Vol. 10, No 7, July 2004, pages 727-734) shows that a certain appetite suppressing effect can be attributed to α-lipoic acid. Therefore, it opens the possibility for the humans of reduced need for taking food due to the absorption of α-lipoic acid, leading thus to reduction in weight.

Accordingly, the aim of the invention is to create a composition of the kind mentioned at the outset which has no side-effects and can contribute to the reduction of weight or can improve such a contribution.

An ubichinon concentrate is known from the document WO 03/007907, which consists of an emulgator and ubichinone Q₁₀, and a light vegetable oil (safflower oil). This concentrate has the property of facilitating the availability of Q₁₀, which is required in the mitochondria for breaking down the fats.

The German Patent Disclosure DE 101 08 614 A1 explains an embodiment for producing a water soluble α-lipoic acid solubilisate consisting of α-lipoic acid and a polysorbate.

To that end, the invention provides a water-free concentrate, which contains the ubichinone Q₁₀, and a medium-chain triglyceride, or a triglyceride mixture and α-lipoic acid or its derivatives and one or more emulgators with a HLB value between 9 and 19 which are permitted according to the food or drug laws. The invention is based on the concept of supporting the decomposition of the fats by supplying a sufficient quantity of Q₁₀ on one hand, and limiting such decomposition to fats stored within the organism, on the other hand, in that simultaneous administration of α-lipoic acid with food intake retards food consumption due to its influence on the hypothalamus. The mentioned ingredients of the concentrate according to the invention are permitted by the food laws and are free of side-effects. The concentrate with suitable proportions by weight of its ingredients is clear and viscous and enables its processing to contents of capsules without problems preferably at slightly higher temperature of about 60° C. Daily administration of such capsules can lead to reduction in the weight of the organism. In the meantime, scientific research carried out has shown that administration of concentrates according to the invention to test persons led to higher percent-wise loss, that is to higher percent-wise loss of visceral fat mass and to greater percent-wise difference in the perimeter of the waist compared to the placebo.

The emulgators usable according to the invention are subject to the respective national and international food or drug regulations. Examples of such solubilizers are the non-ionic polysorbates that are permitted everywhere, above all polysorbate 20 and/or polysorbate 80. For instance, other emulgators are also permitted in the United States of America and in Japan which can also be used in context of the invention.

In addition to the α-lipoic acid, dihydrolipoic acid or dihydrolipoamide can also be used successfully for making a concentrate according to the invention. Further, the compositions preferred according to the invention are mentioned in the dependent claims. In a practical embodiment the concentrate according to the invention contains either only polysorbate 80 or if necessary a mixture of polysorbate 80 with polysorbate 20. Further, it is recommended to use either a mild foodstuff oil such as safflower oil, or a composition, which essentially consists of caprylic acid and caprinic acid, and is available as a product under the trade name Miglyol 812.

The ratio by weight of the polysorbate to the sum of the weights of the ingredients in the concentrate according to the invention lies preferably between about 4:1 and 5.5:1. It is practical if the ratio by weight of Q₁₀ to α-lipoic acid lies between 1:1 to about 1:4 with up to a 20% deviation.

Special exemplary compositions of the concentrate according to the invention are given in the dependent claims.

The concentrate according to the invention is suitable as an additivej to non-alcoholic drinks like water, fruit juice, vegetable juice. A concentration of the concentrate in the drink is recommended between about 1:0.1 up to about 1:5,000. The concentrate can also be added to milk products, honey, plant oils, whereby it is preferred to select the ratio of the concentrate to the mentioned products to between 1:0.1 and about 1:100.

For the production of the concentrate according to the invention, it is practical to proceed in such a manner that at first a solubilizate is obtained from Q₁₀, polysorbate 80 and a medium-chain triglyceride, thereafter a solubilizate is obtained from α-lipoic acid and polysorbate 80 or polysorbate 20, and subsequently the Q₁₀ solubilizate is mixed with α-lipoic acid and stirred to give a homogeneous, clear mass, soluble in water. It is recommended to mix the Q₁₀ solubilizate with α-lipoic acid solubilizate in a ratio by weight of about 2:1, for example, 1.8:1 at a temperature of about 60° C. The optimal solubilization temperature for α-lipoic acid lies significantly higher than that of the heat sensitive Q₁₀, so that separate sediment-free solubilization is recommended at the corresponding temperatures suitable for the two active substances.

Exemplary compositions according to the invention are given in detail in the following.

At the beginning a 5% water-free, water-soluble Q₁₀ solubilizate is prepared as described in the Example 2 of the document WO 03/0077907. According to that Example 790 parts by weight of polysorbate 80 is heated to about 85° C. After that 50 parts of weight of the coenzyme Q₁₀ are added and the mixture (840 parts by weight) is stirred while maintaining the temperature of about 85° C., for some time (about 5 minutes) until the mixture becomes homogeneous and transparent Subsequently, 160 parts by weight of safflower oil is added to this mixture after this oil had been warmed also to about 85° C. After heating, the total mixture is also stirred while maintaining the temperature of about 85° C., for some time (about 2 minutes), until the whole mixture (1,000 parts by weight) also becomes homogeneous and transparent. After cooling to room or body temperature, clearness and water solubility are preserved. One gram of this solubilizate contains 50 mg Q₁₀.

Safflower oil is mentioned as one of the ingredients in that document. Safflower oil may be substituted according to the invention by another medium-chain triglyceride mixture of same quantity, which contains saturated vegetable fatty acids of medium chain-length, consisting essentially of caprylic acid and caprinic acid, and is offered commercially by the Firm Sasol GmbH under the name Miglyol 812 N, for example.

Thereafter, a 10% water-free, water soluble α-lipoic acid solubilizate is prepared by heating at first 900 parts by weight of polysorbate 20 to 60° C. In the warm polysorbate 20, 100 parts by weight of α-lipoic acid are slowly trickled in (CAS No. 62-46-4; ALIPURE of the firm Degussa). Under continuous stirring, the mixture is heated at about 100° C. until it becomes a transparent mixture. On cooling to room temperature, the mixture remains transparent and is fully water soluble in that form. 1 g of this solubilizate contains 100 mg α-lipoic acid. The use of polysorbate 20 facilitates solubilization; but in this case, same quantity of polysorbate 80 is to be preferred due to sensory reasons.

To obtain a Q₁₀-α-Lipoic acid solubilizate, about 660 parts by weight of Q₁₀ solubilizate with about 370 parts by weight of α-lipoic acid solubilizate are stirred at temperature of about 60° C. to yield a homogenous mixture. This mixture contains 33 parts by weight of Q₁₀ and 37 parts by weight of α-lipoic acid, both of which are present in the polysorbate micelles with particle diameter of about 10 nm. With this mixture, gelatin-containing or gelatin-free capsules with 470 mg filling weight are filled up. The content of this capsule consists then of about 15.02 mg Q₁₀, about 16.68 mg α-lipoic acid, about 48.22 mg triglycerides and about 389.7 mg polysorbate 80.

Therefore, on consumption of three capsules of this type per day, the organism takes up about
45.15 mg Q₁₀
50.58 mg α-lipoic acid
144.66 mg triglyceride
1169.1 mg polysorbate 80.

These quantities remain well below the maximum daily doses permitted officially for the respective ingredients according to the food laws.

Further examples for the composition of the concentrate according to the invention are given in the following tables. In the tables, MCT refers to the aforementioned Miglyol 812 and polysorbate refers to polysorbate 80. Preparation of the individual exemplary concentrates is done according to the explanation given in the first example.

Example 2

| | g/kg | w/w % |
|---|---|---|
| A) Q₁₀ | 50 | 5 |
| B) α-lipoic acid | 100 | 10 |
| C) MCT | 40 | 4 |
| D) Polysorbate | 810 | 81 |
| Total: | 1,000 | 100 |

Example 3

| | g/kg | w/w % |
|---|---|---|
| A) Q₁₀ | 40 | 4 |
| B) α-lipoic acid | 80 | 8 |
| C) MCT | 60 | 6 |
| D) Polysorbate | 820 | 82 |
| Total: | 1,000 | 100 |

Example 4

| | g/kg | w/w % |
|---|---|---|
| A) Q₁₀ | 50 | 5 |
| B) α-lipoic acid | 90 | 9 |
| C) MCT | 50 | 5 |
| D) Polysorbate | 810 | 81 |
| Total: | 1,000 | 100 |

Example 5

| | g/kg | w/w % |
|---|---|---|
| A) Q₁₀ | 20 | 2 |
| B) α-lipoic acid | 80 | 8 |
| C) MCT | 60 | 6 |
| D) Polysorbate | 840 | 84 |
| Total: | 1,000 | 100 |

Due to its solubility in water, particularly in mildly warm water (about 35° C.), the appropriate dose of the Q₁₀-α-lipoic acid concentrate according to the invention can be added to alcohol-free drinks, without impairing the clearness of the drink. Further, the concentrate according to the invention can be added to salves or other cosmetic formulations, because the micellar structure of the concentrate facilitates penetration into the skin. Finally, the concentrate according to the invention can be used as a food supplement, or, in higher doses, as a dietary foodstuff.

In the claims:

1. Water-free concentrate, consisting of ubichinon Q₁₀, a medium-chained triglyceride or triglyceride mixture, α-lipoic acid and/or its derivatives, as well as one or more emulgators with HLB value between 9 and 19 permitted according to the food or drug laws.

2. Concentrate according to claim 1, in which the emulgator consists of a polysorbate.

3. Concentrate according to claim 2, in which the emulgator consists of a polysorbate 20 and/or polysorbate 80.

4. Concentrate according to claim 1, in which the α-lipoic acid derivative is dihydrolipoic acid or dihydrolipoamide.

5. Concentrate according to claim 1, in which the medium-chained triglyceride is a light oil such as safflower oil or a mixture of caprylic acid and caprinic acid.

6. Concentrate according to claim 1, in which the ratio by weight of the polysorbate to the sum of the proportions by weight of the remaining ingredients amounts to about 4:1 to about 5.5:1.

7. Concentrate according to claim 1, in which the ratio by weight of ubichinon to α-lipoic acid lies between about 1:1 and about 1:4.

8. Concentrate according to claim 1, which consists of about 85 w/w % of the polysorbates, about 3.3 w/w % of Q₁₀, about 4 w/w % of α-lipoic acid and about 10 w/w % of triglycerides.

9. Concentrate according to claim 1, which consists of about 5 w/w % of Q₁₀, about 10 w/w % of α-lipoic acid, about 4 w/w % of triglycerides and about 81 w/w % of polysorbate 80.

10. Concentrate according to claim 1, which consists of about 4 w/w % of Q₁₀, about 8 w/w % of α-lipoic acid, about 6 w/w % of triglycerides and about 82 w/w % of polysorbate 80.

11. Concentrate according to claim 1, which consists of about 5 w/w % of Q₁₀, about 9 w/w % α-lipoic acid, about 5 w/w % of triglycerides and about 81 w/w % of polysorbate 80.

12. Concentrate according to claim 1, which consists of about 2 w/w % of Q₁₀, about 8 w/w % of α-lipoic acid, about 6 w/w % triglycerides and about 84 w/w % polysorbate 80.

13. Method for production of a concentrate according to claim 1, in which at first a solubilizate is obtained from Q₁₀, polysorbate 80 and a triglyceride, thereafter a solubilizate is obtained from α-lipoic acid and polysorbate 80 or polysorbate 20 and subsequently Q₁₀ solubilizate is mixed with α-lipoic acid solubilizate and stirred to yield a homogeneous mass.

14. Method according to 13, in which an α-lipoic acid solubilsate is obtained at a temperature, for instance 100° C., which is higher compared to that temperature, for instance 85° C., at which Q₁₀ is solubilized, and both the solubilizates are mixed at a lower temperature of, for instance, 60° C.

15. Method according to claim 13, in which about 2, more practically about 1.8 parts of Q₁₀ solubilizate are mixed with about 1 part of α-lipoic acid solubilizate.

16. Concentrate according to claim 1, which is added to a non-alcoholic drink in the ratio of one part of the concentrate to about 0.1 to up to about 5,000 parts of the drink.

17. Concentrate according to claim 1, which is added to a milk product, vegetable oil or a similar foodstuff in the ratio of one part of the concentrate to about 0.1 to up to about 500 parts of the foodstuff.
